(19) United States
(12) Patent Application Publication
(10) Pub. No.: US 2015/0079067 A1
(43) Pub. Date: Mar. 19, 2015

(54) Pharmaceutical composition for repairing wound and/or regenerating tissue, method and uses thereof

(71) Applicant: Taichung Hospital, Ministry of Health and Welfare, Taichung City (TW)

(72) Inventor: Hsin-Chung TSAI, Taichung City (TW)

(73) Assignee: Taichung Hospital, Ministry of Health and Welfare, Taichung City (TW)

(21) Appl. No.: 14/206,222

(22) Filed: Mar. 12, 2014

(30) Foreign Application Priority Data
Sep. 13, 2013 (TW) 102133199

(51) Int. Cl. A61L 26/00

(52) U.S. Cl. CPC A61L 26/0066; A61L 26/0057; A61L 2300/412; A61L 2300/30
USPC 424/94.64; 424/530

(57) ABSTRACT

This invention discloses a method to produce a pharmaceutical composition from bloods for repairing wound which don't have to extra add commercial thrombins or non-human thrombins but to manufacture the pharmaceutical composition from bloods for repairing wound directly from bloods. Therefore, the method discloses in this present invention have the effect to avoid the rejection reaction of the treated patients and to reduce the costs of production, and furthermore to decrease medical costs and the burden of patients.

The current application claims a foreign priority to the patent application of Taiwan No. 102133199 filed on Sep. 13, 2013.

### BACKGROUND OF THE INVENTION

1. Field of the Invention

This invention relates to a method to produce a pharmaceutical composition, specially relates to a method to produce a pharmaceutical composition from bloods for repairing wound and/or regenerating tissue.

2. Description of the Related Art

Platelets can aggregate and stop bleeding in human body and release growth factors at active status. Therefore, platelets play a very important role during the process of hemostasis, tissue repairing and regeneration. Now, the platelet-rich plasma (PRP) is often used to heal wounds at clinical for improving tissue cells regeneration and/or repairing. For example, to use PRP for treating the wound caused by diabetes, festering wound or the patient after operation.

Papers reported that adding sodium citrate as the anticoagulant into patient's bloods, and then centrifuge it to collect plasma, the PRP, at middle layer. Adding normal saline with a predetermined concentration and bovine thrombin into the PRP for promoting growth factors released to improve tissues generation. However, the PRP produced by the mentioned method remains the plasma or the blood to induce immune response at treated patients and then trigger the secondary inflammation. In order to improve the foresaid problem, researchers centrifuge the blood first to get the upper layer liquid and then centrifuge it again to get the middle layer liquid. After that, the middle layer liquid and the thrombins activator are mixed to get a mixture. The mixture is centrifuged and filtered to get the supernatant wherein contains growth factors of the PRP.

Although the method revealed by foresaid research can improve purity of the PRP, but it still lies following problem. First of all, it is high production cost of the PRP. It will become a heavy medical expense for patient to use the PRP for treatment and also to make it difficult for generally uses of the PRP in clinical practice to treat the patient who needs it. Secondly, it takes longer time for production of the PRP. Thirdly, the PRP will clot if adding thrombin too early. Fourthly, the non-autologous enzyme may cause adverse reaction in patients. Fifthly, the yield on production of the PRP is too little to use on the wound with big area.

### SUMMARY OF THE INVENTION

This present invention is related to a method to produce a pharmaceutical composition from bloods for repairing wound. There wouldn't be any extra commercial synthesis thrombin or non-human thrombin but using autologous bloods directly to produce the pharmaceutical composition for repairing wound and/or regenerating tissue. Therefore, according to the method revealed in this present invention, the pharmaceutical composition can medicate on the patient wound to promote tissue regeneration, effectively reduce recovery time and avoid the rejection reaction of the patients.

The major propose of this present invention is to provide a method to produce a pharmaceutical composition from bloods for repairing wound, used for reducing production cost and producing the pharmaceutical composition with large scale.

The first embodiment of this present invention includes following steps:

a. providing at least two tubes of blood, wherein one tube of blood is without the anticoagulant and the other one tube of blood contains the anticoagulant.

b. collecting an upper layer from the tube of blood without the anticoagulant after centrifuging.

c. centrifuging the other one tube of blood containing the anticoagulant and then the blood is divided into at least three layers, wherein the first layer contains erythrocytes, the second layer contains platelet-rich plasma (hereafter referred to as “PRP”) and the third layer contains platelet-poor plasma (hereafter referred to as “PPP”).

d. collecting PRP from the three layers of the step c.

e. collecting PPP from three layers of the step c.

f. mixing the some of upper layer from the step b., the PRP from the step d., the PPP from the step e. and calcium with a predetermined ratio to have a pharmaceutical composition for repairing wound.

In one embodiment of this present invention, the step b. is further to separate the upper layer into a mantle consisting of platelet-rich fibrin (hereafter referred to as “PRF”) and a liquid object consisting of thrombin.

In another embodiment of this present invention, the step d. further comprises a step d1 that is to centrifuge some of the three layers from the step c. to get a layered object and then to collect the PRP from the layered object.

Moreover, the step d1. is to collect the first layer, the second layer, and some of the third layer from the step c. to centrifuge. The step e. is to collect the PPP from the third layer from the step c. and the layered object from the step dl.

According to the embodiments, wherein, in the step f., the liquid object, the PRP from the step d., the PPP from the step e. and calcium are mixed with a predetermined ratio. Furthermore, the volume of the PPP collecting from the step e. is the largest in mixture and the volume of the liquid object, the PRP from the step d. and calcium are the same in mixture. Moreover, the PPP from the step e., the liquid object, the PRP from the step d. and calcium mixed in the volume ratio of 5:1:1:1 or 6:1:1:1 is the best.

The step f. is to mix the some of the PRP from the step d., the PPP from the step e. and calcium first and then adding the liquid object to mix.

Besides, the method disclosed in this present invention further includes adding the mantle into the pharmaceutical composition for repairing wound.

Another propose of this present invention is to provide a method to promote wound repair and/or tissue regeneration, which including following steps:

a. providing a pharmaceutical composition manufactured from the foresaid method provided by this present invention;

b. putting an effective dose of the pharmaceutical composition on a damaged tissue.

In the embodiments of this present invention, the damaged tissue could be bone, cartilage, tendon or full thickness wound.

### BRIEF DESCRIPTION OF THE DRAWINGS

FIG. 1 is a flow chart to illustrate the embodiment of this present invention.

FIG. 2 is a usage diagram to illustrate the embodiment of this present invention.

FIG. 3 is result of the blood containing the anticoagulant after the first centrifugation.

FIG. 4 is result of the blood containing the anticoagulant after twice centrifugation.

FIG. 5A shows the wound resulted from scalp tumor.

FIG. 5B shows the wound resulted from scalp tumor after the debridement operation.

FIG. 5C shows the wound resulted from scalp tumor after treated with the pharmaceutical composition for repairing wound disclosed in this present invention for 6 weeks.

FIG. 6A shows the wound resulted from diabetes.

FIG. 6B shows the wound before the operation.

FIG. 6C shows the wound with the bone exposure in the operation.

FIG. 6D shows the wound resulted from diabetes after treated with the pharmaceutical composition for repairing wound disclosed in this present invention for 4 weeks.

### DETAILED DESCRIPTION OF THE INVENTION

Hereinafter, there is one embodiment of this invention and to illustrate with figures as followings.

Unless defined otherwise, all technical and scientific terms used herein have the same meanings commonly understood by one of ordinary skill in the art. As used in this application, including claims and specification, the following words is only exemplary and illustrative, not limiting in scope.

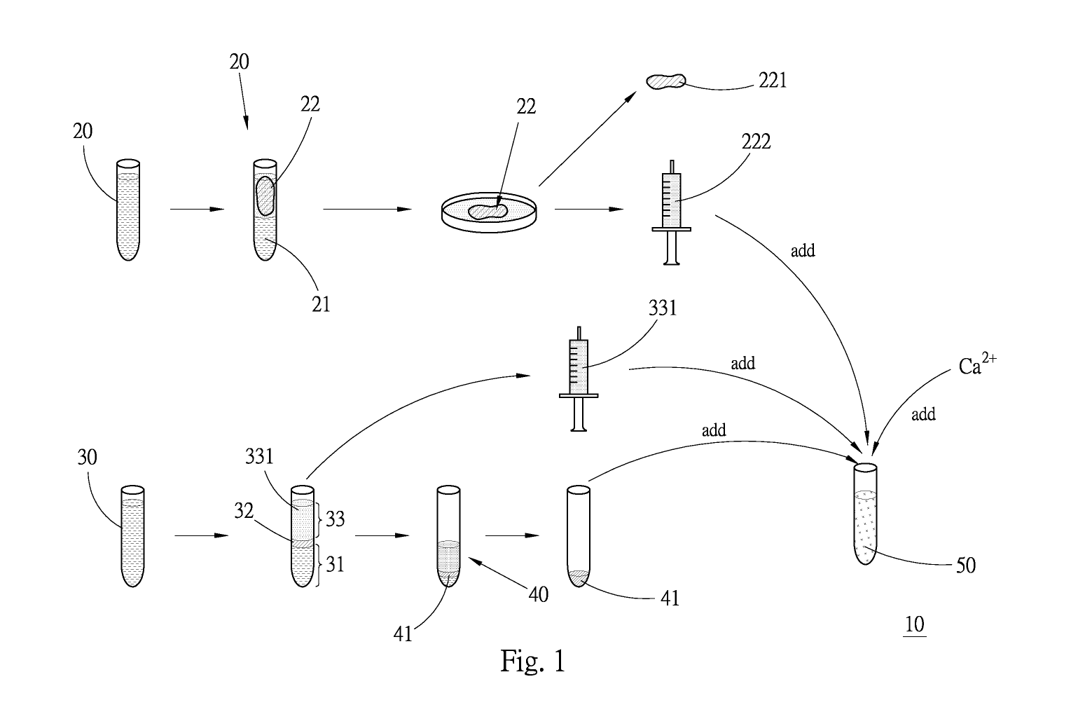

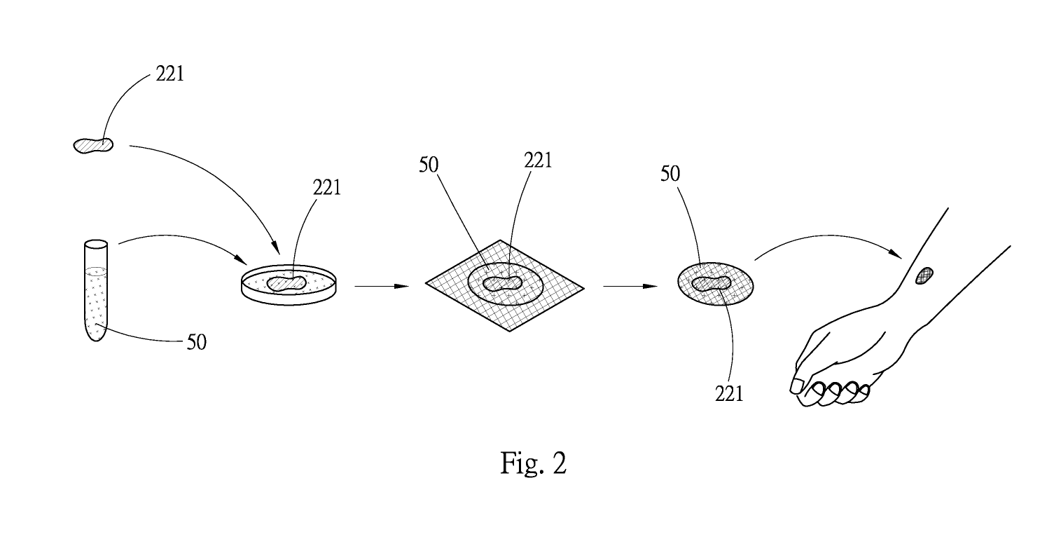

Referring to FIG. 1, the method 10 to produce a pharmaceutical composition for wound repair includes following steps:

Step a. is to provide at least two tubes of blood 20 and 30, wherein one tube of blood 20 is without the anticoagulant and the other one tube of blood 30 contains the anticoagulant.

Step b. is to centrifuge the tube of blood 20 without the anticoagulant to obtain a lower layer 21 and an upper layer 22, wherein the lower layer 21 includes erythrocytes. Collecting the upper layer 22, which is placed in a container, such as a culture dish, and then is pressed for separating into a mantle 221 consisting of PRF and a liquid object 222 consisting of thrombin.

Step c. is to centrifuge the other tube of blood 30 containing the anticoagulant and then the blood in the other tube 30 is divided into three layers 31, 32 and 33, which are as follows from down to up in sequence: a first layer 31 contains erythrocytes, a second layer 32 contains PRP and a third layer 33 contains PPP.

Step d. is to collect some of the first layer 31, the second layer 32, and some of the third layer 33 from step c. and then centrifuging to get a layered object 40.

Step e. is to collect PRP 41 from the layered object of the step d.

Step f. is to collect the PPP 331 from the third layer 33 from the step c.

Step g. is to mix the liquid object 222 from the step b., the PRP 41 from the step e., the PPP 331 from the step f. and calcium with a predetermined ratio to have a pharmaceutical composition 50 for repairing wound.

According to foresaid steps, the pharmaceutical composition 50 for repairing wound produced by the method disclosed in this present invention can be used to contact with wounds to promote wounds regeneration and improve wounds recovery of patients. Furthermore, either to place the pharmaceutical composition 50 for repairing wound on a swab after produced and clotted, then to press till it becomes a membrane and to put the membrane on the wound or to mix the pharmaceutical composition 50 for repairing wound with the mantle 221, then to put the pharmaceutical composition 50 for repairing wound containing the mantle 221 on a swab 50 after it clotted and to press till it becomes a membrane to put on the wound directly to reach the foresaid effect.

To understand the purposes and advantages of the present invention, it is described by the following merely examples taken in conjunction with the accompanying drawings. It will be understood that it is not intended to limit the invention to the described embodiments.

### Example 1

### Production of the Pharmaceutical Composition for Repairing Wound from Bloods

First, 2 tubes of blood which did not contain the anticoagulant from 10 tubes of 8 c.c blood were put symmetrically and levelly into a centrifuge machine and then centrifuge it for about 15 minutes at 3400 rpm. A lower erythrocyte layer was cut by a scissor to keep a light-yellow, gelled upper layer. The gelled upper layer was placed into a small round dish and pressed equally to separate the upper layer into the liquid object consisting of thrombin and the mantle consisting of PRF.

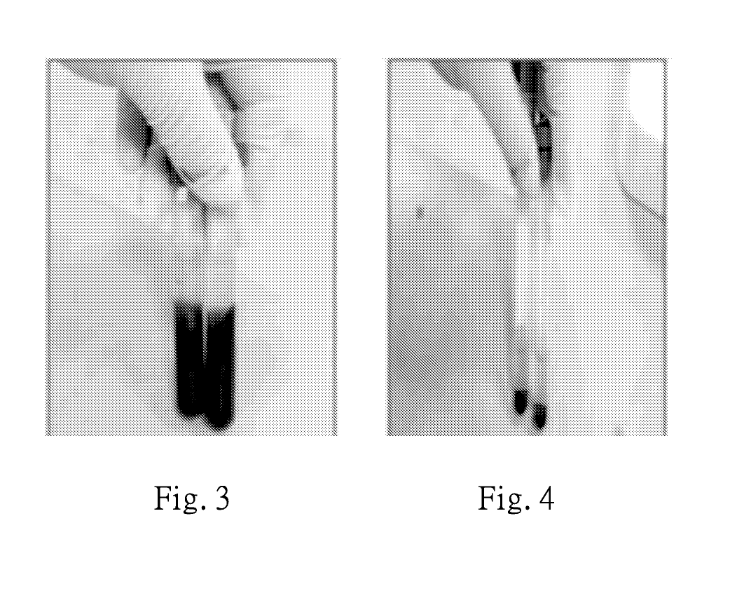

And then, the rest of 8 tubes of blood with the anticoagulant were centrifuged about 5 minutes for the first time. After a lower layer of the each tube which contains the erythrocytes were removed, to collect total volume about 5 mL of the PPP from each tube of blood, then the rest part were centrifuged about 15 minutes for the second time to turn into three different layers. The middle layer with the PRP was collected from the three different layers. Furthermore, the result of the first time centrifugation is showed as FIG. 3 and the result of the second time centrifugation is showed as FIG. 4.

5 mL of the PRP, 1 mL of the middle layer with the PPP and 1 mL of calcium were mixed and then 1 mL of the liquid object of the thrombin was added and mixed well to get the pharmaceutical composition for repairing wound disclosed in this present invention. The mantle of the PRF was then added into the pharmaceutical composition for repairing wound. The pharmaceutical composition for repairing wound containing the PRF was placed on a swap after it clotted and then pressed to become a membrane for use of the following example.

### Example 2

### Examination for Repairing Wounds

In this example, the pharmaceutical composition for repairing wound produced in example 1 and the commercial platelet gel kit were separately covered on the injury site of the patients having an open wound for a predetermined period to observe the recovery progress of each patient. The commercial platelet gel kit included the PRP process device (Registration number: DOH-MD-(I)-No. 001588, Department of Health, Taiwan) and the PPP transfer spike set (Registration number: DOH-MD-(I)-No. 001164, Department of Health, Taiwan).

In detail, there were 39 patients to treat with the pharmaceutical composition for repairing wound disclosed in this present invention and 77 patients to treat with the commercial platelet gel kit. The recovery result of treated patients is showed as the following Table 1.

TABLE 1
The result of different pharmaceutical
composition for repairing wound

| | The present | The commercial |
|---|---|---|
| Pharmaceutical composition | invention | kit |
| Treated patients | 39 | 77 |
| Improved or healed patients | 33 | 63 |
| Ratio (Improved or healed | 84.6% | 81.8% |
| patients/treated patients) | | |

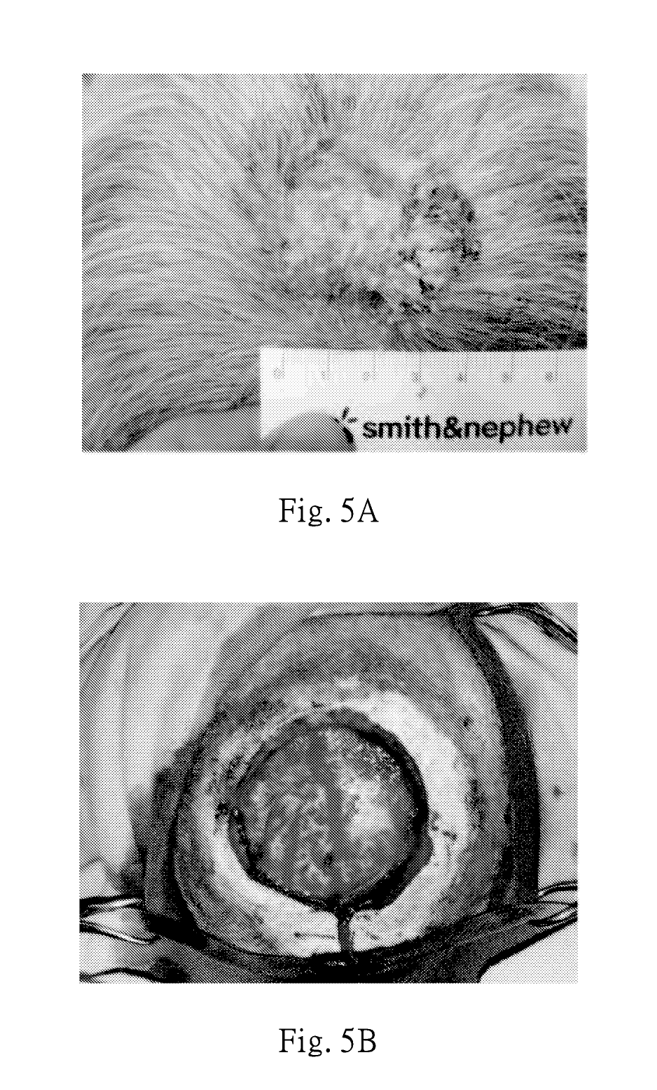

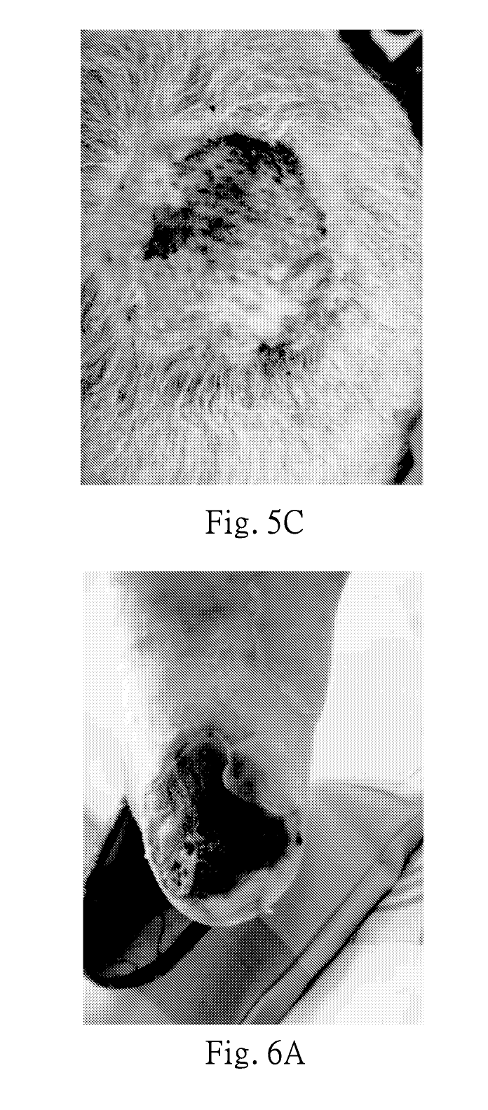

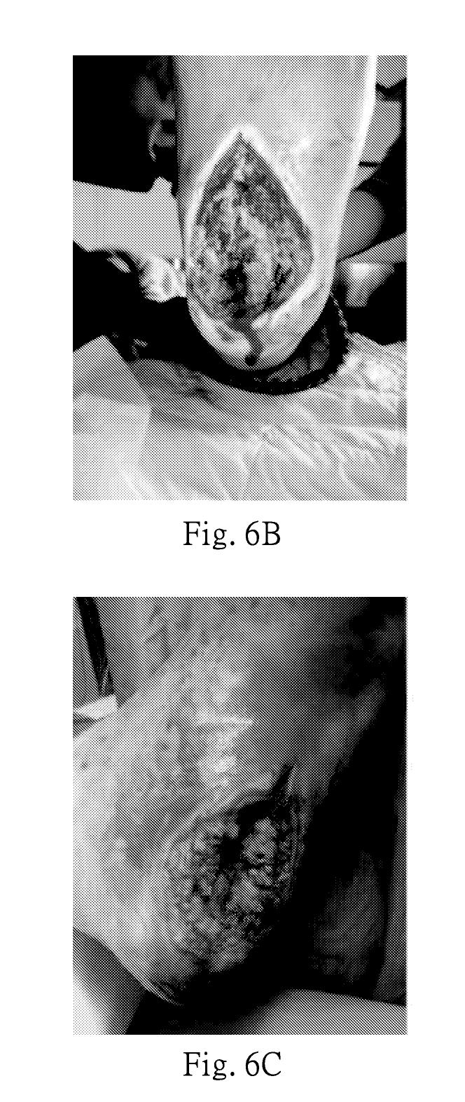

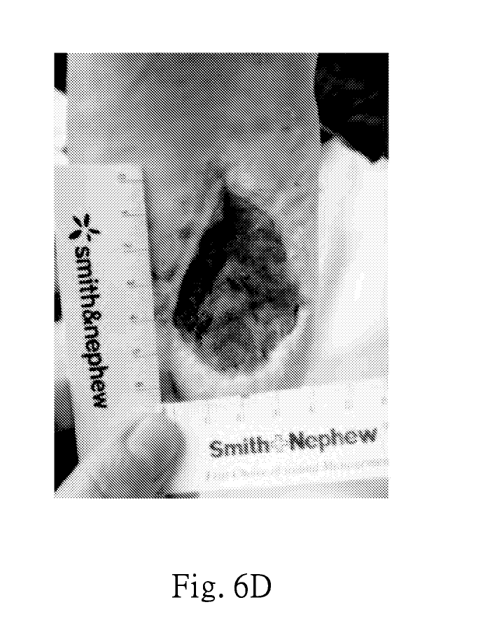

Herein the wound could be resulted from diabetes, tumor, fracture etc. For example, the wound shown in FIG. 5A was resulted from scalp tumor and then underwent a debridement operation in the wound (FIG. 5B). After operation, the wound was treated with the pharmaceutical composition for repairing wound disclosed in this present invention for 6 weeks (FIG. 5C). Moreover, the wound shown in FIG. 6A was resulted from diabetes. Before the operation, the wound was bigger and had bone exposure (FIGS. 6B and 6C). After the operation, the wound was treated with the pharmaceutical composition for repairing wound disclosed in this present invention for 4 weeks, the recovery status of the wound was shown in FIG. 6D.

According to Table 1, FIG. 5 and FIG. 6, it shows that the pharmaceutical composition for repairing wound disclosed in this present invention really can improve the wound recovery and the ratio of recovery patients thereof is higher than the commercial platelet gel kit.

Therefore, the pharmaceutical composition for repairing wound produced by the method of this present invention has the effect to promote tissues generation and improve wounds repair.

The pharmaceutical composition for repairing wound disclosed in this present invention is applicable to every kind of wounds or injured sites which need to promote tissues generation, for example, the bone fractures, full thickness wound etc. The approach of treatment includes not only to cover it directly on the injured site, but also to make it contact the injured sites by the carriers which carry the pharmaceutical composition for repairing wound, to use sterile devices to lead the pharmaceutical composition for repairing wound into the injured sites, or to use any other ways to make the pharmaceutical composition for repairing wound contact the injured sites. In order to operate in coordination for different treatment approach, this present invention of the pharmaceutical composition for repairing wound can be produced into different types, for example, liquid, ointment, gel, powder etc.

To sum up, this present invention of the pharmaceutical composition for repairing wound is manufactured merely by autologous bloods during the process of production without adding non-human or synthesis additives to achieve, so it is for sure that it has the effect of reduce the costs of production and the materials for production of the pharmaceutical composition is able to be from the patients themselves to have the effect that to avoid the rejection reaction of the treated patients.

It should be understood that the above-mentioned detailed description and specific examples, while indicating preferred embodiments of the invention, are given by way of illustration only. Various changes and modifications within the spirit and scope of the invention will become apparent to those skilled in the art from this detailed description. All such modifications as would be obvious to one skilled in the art are intended to be included within the scope of the following claims.

1. A method to manufacture a pharmaceutical composition from bloods for repairing wound and/or regenerating tissue, including following steps:
a. providing at least two tubes of blood, wherein one tube of blood is without the anticoagulant and the other one tube of blood contains the anticoagulant;
b. collecting an upper layer from the tube of blood without the anticoagulant after centrifuging;
c. centrifuging the other one tube of blood containing the anticoagulant divided into at least three layers, wherein the first layer contains erythrocytes, the second layer contains platelet-rich plasma and the third layer contains platelet-poor plasma;
d. collecting the platelet-rich plasma from the three layers of the step c.;
e. collecting the platelet-poor plasma from three layers of the step c.; and
f. mixing some of the upper layer from the step b., the platelet-rich plasma from the step d., the platelet-poor plasma from the step e. and calcium with a predetermined ratio to have a pharmaceutical composition for repairing wound.

2. The method according to claim 1, wherein the step b. is further separated the upper layer into a mantle consisting of platelet-rich fibrin and a liquid object consisting of thrombin.

3. The method according to claim 2, wherein the step f. is to mix the liquid object, the platelet-rich plasma from the step d., the platelet-poor plasma from the step e. and calcium with a predetermined ratio.

4. The method according to claim 3, wherein the volume of the platelet-poor plasma collecting from the step e. is the largest in mixture and the volume of the liquid object, the platelet-rich plasma from the step d. and calcium are the same in mixture.

5. The method according to claim 4, wherein the platelet-poor plasma from the step e., the liquid object, the platelet-rich plasma from the step d. and calcium are mixed in the volume ratio selected from the group consisting of 5:1:1:1 and 6:1:1:1.

6. The method according to claim 3, wherein the step f is to mix the some of the layered object from the step d., the platelet-poor plasma from the step e. and calcium first and then adding the liquid object therein.

7. The method according to claim 3, further includes to add the mantle into the pharmaceutical composition for repairing wound.

8. The method according to claim 1, wherein the step d. further comprises a step dl, centrifuging some of the three layers from the step c. to get a layered object and then collecting the platelet-rich plasma from the layered object.

9. The method according to claim 8, wherein the step d1. is to collect some of the first layer, the second layer, and some of the third layer from the step c. to centrifuge.

10. The method according to claim 8, wherein the step e. is to collect the platelet-poor plasma from the third layer from the step c. and the layered object from the step d1.

11. A method for improving wound healing and/or tissue regeneration, including follow steps:
a. providing a pharmaceutical composition manufactured from the method according to claim 1;
b. putting an effective dose of the pharmaceutical composition on a damaged tissue.

12. The method according to claim 11, wherein the damaged tissue is selected from the group consisting of bone, cartilage, tendon and full thickness wound.
